(19) United States
(12) Patent Application Publication
(10) Pub. No.: US 2006/0020046 A1
(43) Pub. Date: Jan. 26, 2006

(54) Compositions comprising lycopene for the treatment and prevention of angiogenesis associated pathologies

(75) Inventors: Regina Goralczyk, Basel (CH); Klaus Jung, Berlin (DE); Michael Lein, Potsdam (DE); Luca Barella, Basel (CH); Ulrich Siler, Grenzach-Wyhlen (CH); Elisabeth Stocklin, Riehen (CH); Karin Wertz, Rheinfelden (DE)

Correspondence Address: Bryan Cave, 1290 Avenue of the Americas, New York, NY, 10104, US

(73) Assignee: DMS IP ASSETS B.V., TE Heerlen (NL)

(21) Appl. No.: 10/504,829

(22) PCT Filed: Feb. 6, 2003
(86) PCT No.: PCT/EP03/01149

§ 371 Date: Aug. 16, 2004

(30) Foreign Application Priority Data
Feb. 15, 2002 (EP) 02003544.0

(51) Int. Cl. A61K 31/015; A61K 31/21; A61K 31/7048; A61K 31/7008

(52) U.S. Cl. 514/763; 514/514; 514/62; 514/167; 514/456; 514/474; 514/27; 514/458

(57) ABSTRACT

The invention is concerned with the use of lycopene, optionally in combination with vitamin E and/or C or other biologically active ingredients as disclosed in the specification, in the manufacture of a composition for the primary and secondary prevention of angiogenesis-associated pathologies and coadjuvant treatment thereof; as well as with particular novel formulations comprising lycopene.

The present invention relates to the use of lycopene in the prevention and coadjuvant treatment of angiogenesis-associated pathologies. More specifically, the present invention relates to the use of lycopene in the primary prevention (i.e., the prophylactic supplementation of healthy subjects) of the onset of angiogenesis-associated pathologies, in the coadjuvant treatment (i.e. the supplementation accompanying a running therapy of angiogenesis-associated-pathologies) and in the secondary prevention (i.e., the supplementation after a successful therapy for the prevention of relapse) of angiogenesis-associated pathologies.

Angiogenesis, the process of new capillary formation from the preexisting vasculature, is required for successful tumor growth and metastasis. Furthermore, increased neovascularisation is part of the pathology of several non-cancerous diseases, e.g. in chronic inflammations and several eye diseases.

When a primary tumor first arises, proliferation of cancer cells may be balanced by apoptosis, and the tumor may remain undetectable for years until neovascularization appears. Though the relative sudden onset of neovascularization in primary tumors, described as angiogenic switch from avascular to vascular phenotypes, is a discrete event distinct from tumor initiation, unrestricted growth of solid tumors is limited by angiogenesis, as in the absence of access to an adequate vasculature, tumor cells become necrotic and/or apoptotic. For several carcinomas, an elevated serum VEGF level, the major inducer of angiogenesis, is reported, e.g. for epithelial ovarian neoplasms, esophageal squamous cell carcinoma, head and neck carcinoma, lung carcinoma, non-Hodgkin lymphoma, ovarian carcinoma, prostate carcinoma, renal cell carcinoma, and urothelial carcinoma. Furthermore, angiogenesis and the vascular density of tumors have been shown to be associated with tumor metastasis. Several studies reveal that the higher the microvessel count is in areas of highest vessel density, the lower is the rate of overall survival of the tumor patients, see Weidner N, Semple J P, Welch W R, Folkman J. Tumor angiogenesis and metastasis-correlation in invasive breast carcinoma. N Engl J Med (1991) 324:1-8.

Beside cancer, other diseases are also associated with increased neovascularization. In the first acute phase of inflammation, functional changes in the vasculature, such as dilatation, increase in permeability and endothelial activation occur. In the second subacute phase, capillaries and venules remodel with extensive endothelial mitotic activity. Upon chronic stimulation, both increases in capillary density and vascular dilatation can be observed, although these responses can differ significantly between strains of mice and possibly between species. In many chronic inflammatory diseases, e.g. in rheumatoid arthritis or in psoriasis, neovascularization can be identified in the inflamed lesions. These observed pathology accompanying neovascularizations are in line with reports of elevated serum VEGF levels, the major inducer of angiogenesis, in several inflammatory diseases, e.g. inflammatory bowel disease comprising ulcerative colitis and Crohn's disease, inflammatory arthritis (rheumatoid arthritis, self-limiting arthritis and psoriatic arthritis) and osteoarthritis.

Diabetic retinopathy, ischemic retinal-vein occlusion, and retinopathy of prematurity belong to a group of ischemic retinal disorders which are associated with intraocular neovascularization. In neovascular retinopathies, such as proliferative diabetic retinopathy, there is initially extensive active proliferation of new vessels. It has been shown that increased angiogenesis is a central element in these eye pathologies.

Furthermore, neovascularization is a principal cause of visual loss also in the wet form of age-related macular degeneration (AMD), the overall leading cause of blindness.

According to the present invention, it has been found that angiogenesis can be suppressed or inhibited by the administration of lycopene.

The present invention, therefore, is concerned with the use of lycopene in the manufacture of a composition for the primary and secondary prevention of angiogenesis-associated pathologies and coadjuvant treatment thereof. Furthermore, the present invention is concerned with a method of preventing or treating angiogenesis-associated pathologies which comprises administering to a subject in need of such treatment for therapy or prophylaxis an effective amount of lycopene. The present invention is also concerned with certain novel solid galenical formulations comprising lycopene.

In a further and preferred embodiment of the invention, lycopene is used together with vitamin E and/or vitamin C. Most preferred is a combination of lycopene, vitamin E and vitamin C. The term vitamin E as used herein includes racemic vitamin E (D,L-α-tocopherol) or natural vitamin E, as well as derivatives thereof which have biological vitamin E activity, e.g. carboxylic acid esters, such as vitamin E acetate, propionate, butyrate or succinate. The term vitamin C as used herein includes derivatives thereof which have biological vitamin C activity, e.g. esters and salts, such as sodium ascorbate, sodium ascorbyl phosphate, and ascorbyl palmitate. In a further embodiment of the invention, one or more of the following components can be used together with these active ingredients:
(a) Astaxanthin ((3S,3′S)-3,3′-dihydroxy-β,β-carotene-4,4′-dione) and/or one or more isomers and/or monoesters and/or diesters, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid;
(b) β-Carotene and/or one or more isomers thereof;
(c) β-Cryptoxanthin ((3R)-β,β-carotene-3-ol) and/or one or more isomers or esters thereof, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid;
(d) (−)-Epigallocatechin gallate (EGCG) and/or (−)-epicatechin gallate (ECG) and/or one or more derivatives thereof;
(e) Genistein aglycone (4′,5,7-trihydroxyisoflavone) and/or one or more derivatives thereof (genistein glucosides, genistein sulfates, genistein glucuronides);
(f) Lutein ((3R, 3′R, 6′R)-β, ε, carotene-3,3′-diol) and/or one or more isomers and/or monoesters and/or diesters, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid, thereof;
(g) Quercetin (2-(3,4-dihydroxyphenyl)-3,5,7-trihydroxy-4H-1-benzopyrano-4-one) and/or dihydroquercetin and/or one or more derivatives thereof (quercetine glucosides, quercetin glucuronides, quercetine sulphates, methylquercetins (isohamnetin (3′-O-methylquercetin), tamarixetin(4′-O-methylquercetin));
(h) Myricetin and/or one or more derivatives thereof;
(i) Resveratrol (cis-3,4′,5-trihydroxystilbene and/or trans-3,4′,5-trihydroxystilbene) and/or one or more derivatives thereof (resveratrol glucosides, resveratrol sulfates, resveratrol glucuronides);
(j) Rhizoxin and/or one or more derivatives thereof (palmitoyl rhizoxin);
(k) Silymarin (extract from Silybum marianum) and/or one or more derivatives thereof (silymarin dihemisuccinate sodium salt) and/or one or more of its four main components (silybin [synonymous with silibinin, and sometimes incorrectly called silybinin] and/or isosilybin and/or silydianin and/or silychristin) and/or one or more derivatives thereof (silybin-dihemisuccinate, disilybin, silybin-phosphatidylcholine complex, silybin-phosphate);
(l) Vitamin A and/or one or more derivatives thereof (all-trans retinol or all-trans retinyl acetate or all-trans retinyl palmitate);
(m) Vitamin D2 or vitamin D3 or 1α,25-dihydroxyvitamin D3 or 25-hydroxyvitamin D3 or 1α,24R, 25-trihydroxyvitamin D3;
(n) Zeaxanthin ((3R,3′R)-β,β-carotene-3,3′-diol) and/or one or more isomers and stereo-isomers (preferably mesozeaxanthin, 3R,3′S-zeaxanthin) and/or monoesters and/or diesters, preferably esters of saturated alkanoic acids, such as acetic, propionic, palmitic, stearic, and succinic acid, mono-unsaturated fatty acids, such as oleic acid, and poly-unsaturated fatty acids, such as linolic, linoleic, docosahexaenoic, and arachidonic acid, thereof;
(o) Apigenin and/or one or more derivatives thereof;
(p) Carnosic acid and/or one or more derivatives thereof;
(q) Carnosol and/or one or more derivatives thereof;
(r) Depudecin and/or one or more derivatives thereof;
(s) Eponemycin and/or one or more derivatives thereof;
(t) Dihydroeponemycin and/or one or more derivatives thereof;
(u) Epoxomicin and/or one or more derivatives thereof;
(v) Ergosterol and/or one or more derivatives thereof;
(w) Fisetin and/or one or more derivatives thereof;
(x) Fumagillin and/or one or more derivatives thereof;
(y) Lactacystin and/or one or more derivatives thereof;
(z) Luteolin and/or one or more derivatives thereof;
(aa) Motuporamine C and/or one or more derivatives thereof;
(bb) Ovalicin and/or one or more derivatives thereof;
(cc) Radicicol and/or one or more derivatives thereof;
(dd) Curcumin and/or one or more derivatives (demethoxy-curcumin, bis-demethoxycurcumin, sodium curcumionate, bis-demethylcurcumin, tetrahydrocurcumin, diacteylcurcumin, triethylcurcumin) thereof;
(ee) Squalamine and/or one or more derivatives thereof;
(ff) Isoliquiritin, isoliquiritigenin, liquiritigenin and/or one or more derivatives thereof;
(gg) Very-long-chain omega-3 fatty acides (eicosapentaenoic acid [C20: 5, omega-3], decosahexaenoic acid [C22: 6, omega-3], polyunsaturated ω-3 fatty acids);
(hh) Shark cartilage extract.
(ii) Glucosinolate derivatives (Methylsulfinylalkyl glucosinolates [1-methylsulfinylmethyl glucosinolate, 2-methylsulfinylethyl glucosinolate, 3-methylsulfinylpropyl glucosinolate (glucoiberin), 4-methylsulfinylbutyl glucosinolate (glucoraphanin), 5-methylsulfinylpentyl glucosinolate (glucoalysin), 6-methylsulfinylhexyl glucosinolate, 7-methylsulfinylheptyl glucosinolate, 8-methylsulfinyloctyl glucosinolate, 9-methylsulfinylnonyl glucosinolate, 10-methylsulfinyldodecyl glucosinolate] or allyl glucosinolate (sinigrin) or phenylethyl glucosinolate (gluconasturtiin) or 3-butenyl glucosinolate (gluconapin) or indol-3-ylmethyl glucosinolate (glucobrassicin) or derivatives thereof [N-methoxyindol-3-ylmethyl glucosinolate (neoglucobrassicin), 4-hydroxyindol-3-ylmethyl glucosinolate (4-OH glucobrassicin), 4-methoxyindol-3-ylmethyl glucosinolate (4-CH₃O glucobrassicin)]).
(jj) Isothiocyanate derivatives (Methylsulfinylalkyl isothiocyanate [1-methylsulfinylmethyl isothiocyanate, 2-methylsulfinylethyl isothiocyanate, 3-methylsulfinylpropyl isothiocyanate, 4-methylsulfinylbutyl isothiocyanate (sulforaphane), 5-methylsulfinylpentyl isothiocyanate, 6-methylsulfinylhexyl isothiocyanate (6-HITC), 7-methylsulfinylheptyl isothiocyanate, 8-methylsulfinyloctyl isothiocyanate, 9-methylsulfinylnonyl isothiocyanate, 10-methylsulfinyldodecyl isothiocyanate] or allyl isothiocyanate or phenylethyl isothiocyanate (PEITC) or 3-butenyl isothiocyanate or indol-3-ylmethylisothiocyanate or derivatives thereof (N-methoxy indol-3-ylmethylisothiocyanate, 4-hydroxy indol-3-ylmethylisothiocyanate, 4-methoxy indol-3-ylmethylisothiocyanate) or 3-indolmethanol (indol-3-carbinol, I3C).

Examples of angiogenesis associated pathologies comprise Hodgkin lymphoma, non-Hodgkin lymphoma, lymphosarcoma, lymphoblastoid leukemia, acute lymphatic leukemia, acute myeloic leukemia, chronic myeloic leukemia, chronic lymphatic leukemia, hemangioma, hemangioendothelioma, hemangiopericytoma, hemangiosarcoma, Kaposi sarcoma, osteosarcoma, fibrosarcoma, oesophageal squamous cell carcinoma, pancreatic carcinoma, gastrointestinal tumors, colon carcinoma, rectum carcinoma, stomach carcinoma, lymphangiosarcoma, brain tumors, neuroblastoma, schwannoma, pheochromocytoma, lung carcinoma, head and neck squamous cell carcinoma, melanoma, non-melanoma skin carcinoma, leiomyomas, leiomyosarcomas, mammary carcinoma, ovarian cancer, endometrial carcinoma, bladder carcinoma, cervix carcinoma, renal carcinoma, prostate carcinoma, metastasis formation, asthma, rheumatoid arthritis, synovitis, degenerative or inflammatory bone and cartilage destruction, non-rheumatoid arthritis, tendosynovitis, inflammatory pseudotumor, diabetic retinopathy, retinal vein occlusion, age-related macular degeneration, retinopathy of prematurity, choroidal and other intraocular diseases, keratoconjunctivitis, gingivitis, periodontal disease, epulis, gastritis, hepatitis, liver regeneration, chronic pancreatitis, tonsillitis, obesity, leukomalacia, rhinitis, laryngitis, tracheitis, bronchitis, bronchiolitis, pneumonia, interstitial pulmonary fibrosis, neurodermitis, psoriasis, thyroiditis, thyroid enlargement, endometriosis, and glomerulonephritis. Of primary interest for treatment in accordance with the present invention are prostate carcinoma, mammary carcinoma, bladder carcinoma, lung carcinoma, pancreatic carcinoma, melanoma, non-Hodgkin lymphoma, colon/rectum carcinomas, endometrial carcinoma, age-related macular degeneration, prostatitis, diabetic retinopathy, psoriasis, rheumatoid arthritis, non-rheumatoid arthritis and gastritis.

For the primary and secondary prevention and coadjuvant treatment of angiogenesis-associated pathologies in accordance with the present invention lycopene is administered to the subject in need of such treatment, i.e. humans, pets or farm animals in an amount of from about 0.0005 mg/kg body weight to about 5 mg/kg body weight per day. When vitamin E or derivatives thereof is co-administered the daily dosage is from about 0.1 mg/kg body weight to about 15 mg/kg body weight, based on tocopherol. When vitamin C or derivative thereof is co-administered the daily dosage is from about 0.2 mg/kg body weight to about 30 mg/kg body weight, based on ascorbic acid. Other components may be co-administered within dosage ranges set forth below:

| | | | | | |
|---|---|---|---|---|---|
| Astaxanthin | 0.001 | mg/kg | to | 5 | mg/kg |
| β-Carotene | 0.001 | mg/kg | to | 5 | mg/kg |
| β-Cryptoxanthin | 0.001 | mg/kg | to | 5 | mg/kg |
| (−)-epigallocatechin gallate | 0.5 | mg/kg | to | 15 | mg/kg |
| (EGCG) or (−)-epicatechin gallate | | | | | |
| (ECG) or equimolar amounts of | | | | | |
| derivatives | | | | | |
| Genistein aglycone | 0.015 | mg/kg | to | 6 | mg/kg |
| Lutein | 0.001 | mg/kg | to | 5 | mg/kg |
| Quercetin | 0.001 | mg/kg | to | 300 | mg/kg |
| Myricetin | 0.001 | mg/kg | to | 300 | mg/kg |
| Resveratrol | 0.01 | mg/kg | to | 1.5 | mg/kg |
| or equimolar amounts of | | | | | |
| derivatives | | | | | |
| Rhizoxin | 0.001 | mg/kg | to | 20 | mg/kg |
| Palmitoyl Rhizoxin | 0.001 | mg/kg | to | 20 | mg/kg |
| Silymarin | 0.01 | mg/kg | to | 100 | mg/kg |
| Silybin | 0.01 | mg/kg | to | 100 | mg/kg |
| or equimolar amounts of | | | | | |
| derivatives | | | | | |
| Isosilybin | 0.01 | mg/kg | to | 100 | mg/kg |
| or equimolar amounts of | | | | | |
| derivatives | | | | | |
| Silydianin | 0.01 | mg/kg | to | 100 | mg/kg |
| or equimolar amounts of | | | | | |
| derivatives | | | | | |
| Silychristin | 0.01 | mg/kg | to | 100 | mg/kg |
| or equimolar amounts of | | | | | |
| derivatives | | | | | |
| All-trans Retinol | 3 | μg/kg | to | 100 | μg/kg |
| All-trans Retinyl acetate | 3.5 | μg/kg | to | 115 | μg/kg |
| All-trans Retinol palmitate | 5.5 | μg/kg | to | 180 | μg/kg |
| Vitamin D2 (Ergocalciferol) | 0.1 | ng/kg | to | 10 | μg/kg |
| Vitamin D3 (Cholecalciferol) | 0.1 | ng/kg | to | 10 | μg/kg |
| 1α, 25-Dihydroxyvitamin D3 | 0.1 | ng/kg | to | 0.5 | μg/kg |
| 25-Hydroxyvitamin D3 | 0.1 | ng/kg | to | 10 | μg/kg |
| 1α, 24R, 25-Trihydroxyvitamin D3 | 0.1 | ng/kg | to | 0.5 | μg/kg |
| Zeaxanthin | 0.001 | mg/kg | to | 5 | mg/kg |
| Apigenin | 0.01 | mg/kg | to | 500 | mg/kg |
| Carnosic acid | 0.01 | mg/kg | to | 250 | mg/kg |
| Carnosol | 0.01 | mg/kg | to | 250 | mg/kg |
| Depudecin | 0.01 | mg/kg | to | 500 | mg/kg |
| Eponemycin | 0.01 | mg/kg | to | 500 | mg/kg |
| Dihydroeponemycin | 0.01 | mg/kg | to | 500 | mg/kg |
| Epoxomicin | 0.01 | mg/kg | to | 500 | mg/kg |
| Ergosterol | 0.1 | mg/kg | to | 2000 | mg/kg |
| Fisetin | 0.01 | mg/kg | to | 500 | mg/kg |
| Fumagillin | 0.1 | mg/kg | to | 300 | mg/kg |
| Lactacystin | 0.01 | mg/kg | to | 250 | mg/kg |
| Luteolin | 0.01 | mg/kg | to | 100 | mg/kg |
| Motuporamine C | 0.1 | mg/kg | to | 500 | mg/kg |
| Ovalicin | 0.1 | mg/kg | to | 250 | mg/kg |
| Radicicol | 0.1 | mg/kg | to | 1000 | mg/kg |
| Curcumin | 0.1 | mg/kg | to | 200 | mg/kg |
| or equimolar amounts of | | | | | |
| derivatives | | | | | |
| Squalamine | 0.001 | | to | 200 | mg/kg |
| Isoliquiritin | 1 | ng/kg | to | 1 | mg/kg |
| Isoliquiritigenin | 1 | ng/kg | to | 1 | mg/kg |
| Very-long-chain omega-3 fatty | 0.001 | g/kg | to | 0.05 | g/kg |
| acides | | | | | |
| Shark cartilage extract | 0.001 | g/kg | to | 0.1 | g/kg |
| Glucosinolate derivatives | 0.01 | mg/kg | to | 200 | mg/kg |
| e.g. 4-methylsulfinylbutyl gluco- | | | | | |
| sinolate (glucoraphanin) | | | | | |
| Isothiocyanate derivatives or I3C | 0.001 | mg/kg | to | 200 | mg/kg |
| e.g. 4-methylsulfinylbutyl | | | | | |
| isothiocyanate (sulforaphane) | | | | | |

Lycopene, optionally together with the vitamins E and C as well as compounds (a) to (jj) can find use in accordance with the present invention for the completion of human nutrition, nutrition of pets and farm animals.

Said compounds may be provided as the active ingredient in compositions, preferably for enteral application, which may be solid or liquid galenical formulations, dietary compositions or animal feed compositions. Examples of solid galenical formulations are tablets, capsules (e.g. hard or soft shell gelatin capsules), pills, sachets, powders, granules and the like which contain the active ingredient together with conventional galenical carriers. Any conventional carrier material can be utilized. The carrier material can be organic or inorganic inert carrier material suitable for oral administration. Suitable carriers include water, gelatin, gum arabic, lactose, starch, magnesium stearate, talc, vegetable oils, and the like. Additionally, additives such as flavouring agents, preservatives, stabilizers, emulsifying agents, buffers and the like may be added in accordance with accepted practices of pharmaceutical compounding. They may also be used in dietary compositions which may be a food, a food premix or a fortified food or a beverage. While the individual active ingredients are suitably administered in a single composition they may also be administered in individual dosage units.

Preferably lycopene is used in accordance with the present invention together with vitamin E or vitamin E and vitamin C. Preferred additional components are the active ingredients (a), (b), (c), (d), (e), (f), (g), (h), (i), (j), (k), (l), (m), and/or (n); more preferably the active ingredients (b), (d), (e), (f), (g), (h), (i), (j), (k), and/or (n).

Particularly preferred is the administration of the following active ingredients:

Lycopene, in a concentration so that the daily consumption by a human adult is in the range of from 0.25 mg/day to 50 mg/day, preferably from 1 mg/day to 30 mg/day; and/or

Vitamin E or its derivative, in a concentration so that the daily consumption by a human adult is in the range of from 15 mg/day to 600 mg/day; and/or

Vitamin C or its derivative, in a concentration so that the daily consumption by a human adult is in the range of from 50 mg/day to 1000 mg/day; and/or

β-Carotene, in a concentration so that the daily consumption by a human adult is in the range of from 0.1 mg/day to 20 mg/day, preferably from 2 mg/day to 10 mg/day; and/or

(−)-Epigallocatechin gallate (EGCG), in a concentration so that the daily consumption by a human adult is in the range of from 50 mg/day to 500 mg/day; and/or

Genistein, in a concentration so that the daily consumption by a human adult is in the range of from 20 mg/day to 200 mg/day; and/or

Lutein, in a concentration so that the daily consumption by a human adult is in the range of from 0.1 mg/day to 50 mg/day, preferably from 0.25 mg/day to 30 mg/day; and/or

Quercetin, in a concentration so that the daily consumption by a human adult is in the range of from 1 mg/day to 500 mg/day; and/or

Myricetin, in a concentration so that the daily consumption by a human adult is in the range of from 1 mg/day to 500 mg/day; and/or

Resveratrol, in a concentration so that the daily consumption by a human adult is in the range of from 5 mg/day to 50 mg/day; and/or

Silymarin (extract from Silybum marianum) or its four main components (silybin and/or isosilybin and/or silydianin and/or silychristin), in a concentration so that the daily consumption by a human adult of Silymarin or its four main components (silybin, isosilybin, silydianin, silychristin), respectively, is in the range of from 1 mg/day to 1000 mg/day, preferably from 50 mg/day to 800 mg/day; and/or

Zeaxanthin, in a concentration so that the daily consumption by a human adult is in the range of from 0.1 mg/day to 50 mg/day, preferably from 0.25 mg/day to 30 mg/day.

Typical examples of galenical formulations for use in accordance with the present invention are given below. The Examples are for the purpose of illustrating the invention and are not intended to limit the scope of the invention in any way.

### EXAMPLE 1

A tablet for the coadjuvant treatment of prostate carcinoma is formulated to contain 5 mg of lycopene, 200 mg of vitamin E, 250 mg of vitamin C, 37.5 mg of resveratrol, and 50 mg of quercetin. The daily dose corresponds to two such tablets.

### EXAMPLE 2

A tablet for the primary prevention of gastritis is formulated to contain 3.5 mg of lycopene, 150 mg of vitamin E, 100 mg of vitamin C, 25 mg of resveratrol, 2.5 mg of lutein and 3.5 mg of β-carotene. The daily dose corresponds to two such tablets.

### EXAMPLE 3

A tablet for the primary prevention of age-related macular degeneration is formulated to contain 3.5 mg of lycopene, 50 mg of vitamin E, 50 mg of vitamin C, 5 mg of lutein, 5 mg of zeaxanthin and 5 mg of β-carotene. The daily dose corresponds to two such tablets.

### EXAMPLE 4

A patient weighing 70 kg and receiving conventional prostate carcinoma therapy is administered, for the duration of carcinoma therapy, 10 mg of lycopene, 200 mg of vitamin E, 250 mg of vitamin C, 37.5 mg of resveratrol, and 50 mg of quercetin per day in a single dosage unit, e.g. by administration of 2 tablets of Example 1, or in individual dosage units of the components.

### EXAMPLE 5

A patient weighing 70 kg with a history of episodes of gastritis is administered, prophylactically, 7 mg of lycopene, 300 mg of vitamin E, 200 mg of vitamin C, 50 mg of resveratrol, 5 mg of lutein and 7 mg of β-carotene per day in a single dosage unit, e.g. by administration of 1 tablet of Example 1, or in individual dosage units of the components.

### EXAMPLE 6

A patient weighing 70 kg who is prone to age-related macular degeneration is administered 7 mg of lycopene, 100 mg of vitamin E, 100 mg of vitamin C, 10 mg of lutein, 10 mg of zeaxanthin and 10 mg of β-carotene per day in a single dosage unit, e.g. by administration of 1 tablet of Example 1, or in individual dosage units of the components.

1. A method of manufacture of a composition for the primary and secondary prevention of a angiogenesis-associated pathology and coadjuvant treatment thereof comprising admixing lycopene with a carrier material such that the amount of lycopene in the composition which is manufactured is effective to treat or prevent the angiogenesis-associated pathology.

2. A method as in claim 1 further comprising admixing vitamin E with the carrier material.

3. A method as in claim 1 further comprising admixing vitamin E and vitamin C with the carrier material.

4. A method according to claim 1 further comprising admixing one or more compounds selected from the group consisting of β-carotene, (−)-epigallocatechin gallate, genistein, lutein, quercetin, myricetin, resveratrol, silymarin or its four main components (silybin and/or isosilybin and/or silydianin and/or silychristin), and zeaxanthin with the carrier material.

5. A method according to claim 1 further comprising admixing one or more compounds selected from the group consisting of astaxanthin, β-carotene, β-cryptoxanthin, (−)-epigallocatechin gallate (EGCG) and (−)-epicatechin gallate (ECG) and equimolar amounts of derivatives, genistein aglycone, lutein, quercetin, myricetin, resveratrol and equimolar amounts of derivatives, rhizoxin, palmitoyl rhizoxin, silymarin, silybin and equimolar amounts of derivatives, isosilybin and equimolar amounts of derivatives, silydianin and equimolar amounts of derivatives, silychristin and equimolar amounts of derivatives, all-trans retinol, all-trans retinyl acetate, all-trans retinol palmitate, vitamin D2 (ergocalciferol), vitamin D3, (cholecalciferol), 1α,25-dihydroxyvitamin D3, 25-hydroxyvitamin D3, 1α,24R,25-trihydroxyvitamin D3, zeaxanthin, apigenin, carnosic acid, carnosol, depudecin, eponemycin, dihydroeponemycin, epoxomicin, ergosterol, fisetin, fumagillin, lactacystin, luteolin, motuporamine C, ovalicin, radicicol, curcumin and equimolar amounts of derivatives, squalamine, isoliquiritin, isoliquiritigenin, very-long-chain omega-3 fatty acides, shark cartilage extract, glucosinolate derivatives: methylsulfinylalkyl glucosinolates (1-methylsulfinylmethyl glucosinolate, 2-methylsulfinylethyl glucosinolate, 3-methylsulfinylpropyl glucosinolate (glucoiberin), 4-methylsulfinylbutyl glucosinolate (glucoraphanin), 5-methylsulfinylpentyl glucosinolate (glucoalysin), 6-methylsulfinylhexyl glucosinolate, 7-methylsulfinylheptyl glucosinolate, 8-methylsulfinyloctyl glucosinolate, 9-methylsulfinylnonyl glucosinolate, 10-methylsulfinyldodecyl glucosinolate) and allyl glucosinolate (sinigrin) and indol-3-ylmethyl glucosinolate (glucobrassicin) and derivatives thereof, (N-methoxyindol-3-ylmethyl glucosinolate (neoglucobrassicin), 4-hydroxyindol-3-ylmethyl glucosinolate (4-OH glucobrassicin), 4-methoxyindol-3-ylmethyl glucosinolate (4-CH₃O glucobrassicin)) and phenylethyl glucosinolate (gluconasturtiin) and 3-butenyl glucosinolate (gluconapin)), isothiocyanate derivatives: methylsulfinylalkyl isothiocyanates (1-methylsulfinylmethyl isothiocyanate, 2-methylsulfinylethyl isothiocyanate, 3-methylsulfinylpropyl isothiocyanate, 4-methylsulfinylbutyl isothiocyanate (sulforaphane), 5-methylsulfinylpentyl isothiocyanate, 6-methylsulfinylhexyl isothiocyanate (6-HITC), 7-methylsulfinylheptyl isothiocyanate, 8-methylsulfinyloctyl isothiocyanate, 9-methylsulfinylnonyl-isothiocyanate, 10-methylsulfinyldodecyl isothiocyanate) and allyl isothiocyanate, indol-3-ylmethylisothiocyanate, N-methoxy indol-3-ylmethylisothiocyanate, 4-hydroxy indol-3-ylmethylisothiocyanate, 4-methoxy indol-3-ylmethylisothiocyanate, 3-indolmethanol, phenylethyl isothiocyanate (PEITC), and 3-butenyl isothiocyanate with the carrier material.

6. A method according to claim 1 wherein the angiogenesis-associated pathology is selected from the group consisting of Hodgkin lymphoma, non-Hodgkin lymphoma, lymphosarcoma, lymphoblastoid leukemia, acute lymphatic leukemia, acute myeloic leukemia, chronic myeloic leukemia, chronic lymphatic leukemia, hemangioma, hemangioendothelioma, hemangiopericytoma, hemangiosarcoma, Kaposi sarcoma, osteosarcoma, fibrosarcoma, oesophageal squamous cell carcinoma, pancreatic carcinoma, gastrointestinal tumors, colon carcinoma, rectum carcinoma, stomach carcinoma, lymphangiosarcoma, brain tumors, neuroblastoma, schwannoma, pheochromocytoma, lung carcinoma, head and neck squamous cell carcinoma, melanoma, non-melanoma skin carcinoma, leiomyomas, leiomyosarcomas, mammary carcinoma, ovarian cancer, endometrial carcinoma, bladder carcinoma, cervix carcinoma, renal carcinoma, prostate carcinoma, metastasis formation, asthma, rheumatoid arthritis, synovitis, degenerative or inflammatory bone and cartilage destruction, non-rheumatoid arthritis, tendosynovitis, inflammatory pseudotumor, diabetic retinopathy, retinal vein occlusion, age-related macular degeneration, retinopathy of prematurity, choroidal and other intraocular diseases, keratoconjunctivitis, gingivitis, periodontal disease, epulis, gastritis, hepatitis, liver regeneration, chronic pancreatitis, tonsillitis, obesity, leukomalacia, rhinitis, laryngitis, tracheitis, bronchitis, bronchiolitis, pneumonia, interstitial pulmonary fibrosis, neurodermitis, psoriasis, thyroiditis, thyroid enlargement, endometriosis, and glomerulonephritis.

7. A method according to claim 1 wherein the angiogenesis-associated pathology is selected from the group consisting of prostate carcinoma, mammary carcinoma, bladder carcinoma, lung carcinoma, pancreatic carcinoma, melanoma, non-Hodgkin lymphoma, colon/rectum carcinomas, endometrial carcinoma, age-related macular degeneration, prostatitis, diabetic retinopathy, psoriasis, rheumatoid arthritis, non-rheumatoid arthritis and gastritis.

8. A method according to claim 1 wherein the angiogenesis-associated pathology is prostate carcinoma.

9. A method according to claim 1 wherein the angiogenesis-associated pathology is mammary carcinoma.

10. A method according to claim 1 wherein the angiogenesis-associated pathology is gastritis.

11. A method according to claim 1 wherein the angiogenesis-associated pathology is age-related macular degeneration.

12. A method according to claim 1 wherein the composition is a solid or liquid galenical formulation, a dietary composition or an animal feed composition.

13. A method as in claim 12 wherein a dosage unit of said solid galenical formulation contains from about 0.25 mg to about 50 mg of lycopene.

14. A method as in claim 12 wherein said liquid galenical formulation contains from about 0.1 mg to about 100 mg of lycopene per ml.

15. A method as in claim 12 wherein said dietary composition or animal feed composition contains from about 0.025 mg to about 5 mg of lycopene per g.

16. A method as in claim 13 wherein a dosage unit of said solid galenical formulation further contains from about 15 mg to about 500 mg of vitamin E.

17. A method as in claim 14 wherein said liquid galenical formulation further contains from about 10 mg to about 300 mg of vitamin E per ml.

18. A method as in claim 15 wherein said dietary composition or animal feed composition further contains from about 1.5 mg to about 30 mg of vitamin E per g.

19. A method as in claim 13 wherein a dosage unit of said solid galenical formulation further contains from about 50 mg to about 500 mg of vitamin C.

20. A method as in claim 14 wherein said liquid galenical formulation further contains from about 50 mg to about 100 mg of vitamin C per ml.

21. A method as in claim 15 wherein said dietary composition or animal feed composition further contains from about 5 mg to about 50 mg of vitamin C per g.

22. A method according to claim 1 wherein the composition further contains one or more active ingredients selected from the group consisting of β-carotene, (−)-epigallocatechin gallate, genistein, lutein, quercetin, resveratrol, silymarin or one or more of its four main components (silybin and/or isosilybin and/or silydianin and/or silychristin), and zeaxanthin.

23. A method according to claim 1 wherein the composition further contains one or more active ingredients selected from the group consisting of astaxanthin, β-carotene, β-cryptoxanthin, (−)-epigallocatechin gallate (EGCG) and (−)-epicatechin gallate (ECG) and equimolar amounts of derivatives, genistein aglycone, lutein, quercetin, myricetin, resveratrol and equimolar amounts of derivatives, rhizoxin, palmitoyl rhizoxin, silymarin, silybin and equimolar amounts of derivatives, isosilybin and equimolar amounts of derivatives, silydianin and equimolar amounts of derivatives, silychristin and equimolar amounts of derivatives, all-trans retinol, all-trans retinyl acetate, all-trans retinol palmitate, vitamin D2 (ergocalciferol), vitamin D3, (cholecalciferol), 1α,25-dihydroxyvitamin D3, 25-hydroxyvitamin D3, 1α,24R,25-trihydroxyvitamin D3, zeaxanthin, apigenin, carnosic acid, carnosol, depudecin, eponemycin, dihydroeponemycin, epoxomicin, ergosterol, fisetin, fumagillin, lactacystin, luteolin, motuporamine C, ovalicin, radicicol, curcumin and equimolar amounts of derivatives, squalamine, isoliquiritin, isoliquiritigenin, very-long-chain omega-3 fatty acides, shark cartilage extract, glucosinolate derivatives: methylsulfinylalkyl glucosinolates (1-methylsulfinylmethyl glucosinolate, 2-methylsulfinylethyl glucosinolate, 3-methylsulfinylpropyl glucosinolate (glucoiberin), 4-methylsulfinylbutyl glucosinolate (glucoraphanin), 5-methylsulfinylpentyl glucosinolate (glucoalysin), 6-methylsulfinylhexyl glucosinolate, 7-methylsulfinylheptyl glucosinolate, 8-methylsulfinyloctyl glucosinolate, 9-methylsulfinylnonyl glucosinolate, 10-methylsulfinyldodecyl glucosinolate) and allyl glucosinolate (sinigrin) and indol-3-ylmethyl glucosinolate (glucobrassicin) and derivatives thereof, (N-methoxyindol-3-ylmethyl glucosinolate (neoglucobrassicin), 4-hydroxyindol-3-ylmethyl glucosinolate (4-OH glucobrassicin), 4-methoxyindol-3-ylmethyl glucosinolate (4-CH₃O glucobrassicin)) and phenylethyl glucosinolate (gluconasturtiin) and 3-butenyl glucosinolate (gluconapin)), isothiocyanate derivatives: methylsulfinylalkyl isothiocyanates (1-methylsulfinylmethyl isothiocyanate, 2-methylsulfinylethyl isothiocyanate, 3-methylsulfinylpropyl isothiocyanate, 4-methylsulfinylbutyl isothiocyanate (sulforaphane), 5-methylsulfinylpentyl isothiocyanate, 6-methylsulfinylhexyl isothiocyanate (6-HITC), 7-methylsulfinylheptyl isothiocyanate, 8-methylsulfinyloctyl isothiocyanate, 9-methylsulfinylnonyl isothiocyanate, 10-methylsulfinyldodecyl isothiocyanate) and allyl isothiocyanate, indol-3-ylmethylisothiocyanate, N-methoxy indol-3-ylmethylisothiocyanate, 4-hydroxy indol-3-ylmethylisothiocyanate, 4-methoxy indol-3-ylmethylisothiocyanate, 3-indolmethanol, phenylethyl isothiocyanate (PEITC), and 3-butenyl isothiocyanate.

24. A method according to claim 1 for the manufacture of a solid galenical formulation for the coadjuvant treatment of prostate carcinoma containing lycopene in combination with vitamin E, vitamin C, resveratrol, and quercetin in one dosage unit.

25. A method according to claim 1 for the manufacture of a solid galenical formulation for the primary prevention of gastritis containing lycopene in combination with vitamin E, vitamin C, resveratrol, and P-carotene in one dosage unit.

26. A method according to claim 1 for the manufacture of a solid galenical formulation for the primary prevention of age-related macular degeneration containing lycopene in combination with vitamin E, vitamin C, lutein, zeaxanthin, and β-carotene in one dosage unit.

27. A solid galenical formulation containing per dosage unit (with 2 dosage units per day) 5 mg of lycopene, 200 mg of vitamin E, 250 mg of vitamin C, 37.5 mg of resveratrol, and 50 mg of quercetin.

28. A solid galenical formulation containing per dosage unit (with 2 dosage units per day) 3.5 mg of lycopene, 150 mg of vitamin E, 100 mg of vitamin C, 25 mg of resveratrol, 2.5 mg of lutein and 3.5 mg of β-carotene.

29. A solid galenical formulation containing per dosage unit (with 2 dosage units per day) 3.5 mg of lycopene, 50 mg of vitamin E, 50 mg of vitamin C, 5 mg of lutein, 5 mg of zeaxanthin and 5 mg of β-carotene.

30. A method of preventing or treating an angiogenesis-associated pathology which comprises administering to a subject in need of such treatment for therapy or prophylaxis an effective amount of lycopene.

31. A method as in claim 30 wherein the angiogenesis-associated pathology is selected from the group consisting of Hodgkin lymphoma, non-Hodgkin lymphoma, lymphosarcoma, lymphoblastoid leukemia, acute lymphatic leukemia, acute myeloic leukemia, chronic myeloic leukemia, chronic lymphatic leukemia, hemangioma, hemangioendothelioma, hemangiopericytoma, hemangiosarcoma, Kaposi sarcoma, osteosarcoma, fibrosarcoma, oesophageal squamous cell carcinoma, pancreatic carcinoma, gastrointestinal tumors, colon carcinoma, rectum carcinoma, stomach carcinoma, lymphangiosarcoma, brain tumors, neuroblastoma, schwannoma, pheochromocytoma, lung carcinoma, head and neck squamous cell carcinoma, melanoma, non-melanoma skin carcinoma, leiomyomas, leiomyosarcomas, mammary carcinoma, ovarian carcinoma, endometrial carcinoma, bladder carcinoma, cervix carcinoma, renal carcinoma, prostate carcinoma, metastasis formation, asthma, rheumatoid arthritis, synovitis, degenerative or inflammatory bone and cartilage destruction, non-rheumatoid arthritis, tendosynovitis, inflammatory pseudotumor, diabetic retinopathy, retinal vein occlusion, age-related macular degeneration, retinopathy of prematurity, choroidal and other intraocular diseases, keratoconjunctivitis, gingivitis, periodontal disease, epulis, gastritis, hepatitis, liver regeneration, chronic pancreatitis, tonsillitis, obesity, leukomalacia, rhinitis, laryngitis, tracheitis, bronchitis, bronchiolitis, pneumonia, interstitial pulmonary fibrosis, neurodermitis, psoriasis, thyroiditis, thyroid enlargement, endometriosis, and glomerulonephritis.

32. A method as in claim 30 wherein the angiogenesis-associated pathology is selected from the group consisting of prostate carcinoma, mammary carcinoma, bladder carcinoma, lung carcinoma, pancreatic carcinoma, melanoma, non-Hodgkin lymphoma, colon/rectum carcinomas, endometrial carcinoma, age-related macular degeneration, prostatitis, diabetic retinopathy, psoriasis, rheumatoid arthritis, non-rheumatoid arthritis, and gastritis.

33. A method as in claim 30 wherein the angiogenesis-associated pathology is prostate carcinoma.

34. A method as in claim 30 wherein the angiogenesis-associated pathology is mammary carcinoma.

35. A method as in claim 30 wherein the angiogenesis-associated pathology is gastritis.

36. A method as in claim 30 wherein the angiogenesis-associated pathology is age-related macular degeneration.

37. A method as in claim 30 wherein about 0.25 mg to about 50 mg of lycopene are administered per day to a human adult.

38. A method as in claim 30 wherein about 1 mg to about 30 mg of 20 lycopene are administered per day to a human adult.

39. A method as in claim 30 wherein, additionally, about 15 mg to about 600 mg of vitamin E are administered per day to a human adult.

40. A method as in claim 30 wherein, additionally, about 50 to about 1000 mg of vitamin C are administered per day to a human adult.

41. A method for the coadjuvant treatment of prostate carcinoma which comprises administering to a human adult lycopene in combination with vitamin E, vitamin C, resveratrol, and quercetin.

42. A method for the primary prevention of gastritis which comprises administering to a human adult lycopene, vitamin E, vitamin C, resveratrol, lutein, and β-carotene.

43. A method for the primary prevention of age-related macular degeneration which comprises administering to a human adult lycopene, vitamin E, vitamin C, lutein, zeaxanthin, and β-carotene.

44. A method as in claim 39, wherein 10 mg of lycopene, 400 mg of vitamin E, 500 mg of vitamin C, 75 mg of resveratrol, and 100 mg of quercetin are administered to a human adult per day.

45. A method as in claim 40, wherein 7 mg of lycopene, 300 mg of vitamin E, 200 mg of vitamin C, 50 mg of resveratrol, 5 mg of lutein, and 7 mg of β-carotene are administered to a human adult per day.

46. A method as in claim 41, wherein 7 mg of lycopene, 100 mg of vitamin E, 100 mg of vitamin C, 10 mg of lutein, 10 mg of zeaxanthin, and 10 mg of β-carotene are administered to a human adult per day.

47. (canceled)
